(19) United States
(12) Patent Application Publication
(10) Pub. No.: US 2016/0183776 A1
(43) Pub. Date: Jun. 30, 2016

(54) WIRELESS TRANSMISSION UNIT FOR DENTAL INSTRUMENT WITH BUILT-IN CAMERA, AND DENTAL INSTRUMENT WITH BUILT-IN CAMERA

(71) Applicant: THE YOSHIDA DENTAL MFG. CO., LTD., Tokyo (JP)

(72) Inventors: Michizo YAMANAKA, Tokyo (JP); Takeshi HASEGAWA, Tokyo (JP); Terumi TAKEMOTO, Tokyo (JP); Satoru KUMACHI, Tokyo (JP); Hideki MOGI, Tokyo (JP)

(21) Appl. No.: 14/909,657

(22) PCT Filed: Aug. 1, 2014
(86) PCT No.: PCT/JP2014/070315

§ 371 Date: Feb. 2, 2016

(30) Foreign Application Priority Data
Aug. 2, 2013 (JP) 2013-161417

(51) Int. Cl. A61B 1/247; A61B 1/018; A61C 3/02; A61C 17/20; H04N 7/18; A61C 1/05

(52) U.S. Cl. CPC A61B 1/247; H04N 7/183; A61C 1/05; A61C 3/02; A61C 17/20; A61B 1/018

(57) ABSTRACT

The present invention provides a wireless transmission unit for use in a dental instrument with a built-in camera, which is capable of wirelessly transmitting camera images to various destination apparatuses and achieving an improvement in visibility of an affected part while improving operability for a dentist or the like during treatment, and the promotion of patient understanding of the contents of treatment.

A wireless transmission unit (51) of the present invention is configured to have a mounting cylinder (55) having a cylindrical connector section (53) which is fitted on an outer periphery of a hand piece main section (2) of a camera built-in hand piece (1) which is a dental instrument with a built-in camera, and in which the other end of a connection wiring part having one end connected to a built-in camera unit (31) is led outside the hand piece main section (2) so that the cylindrical connector section is connected to the other end; and an image processing wireless transmission means (58) which has a linking connector part (56) linked to the cylindrical connector section (53) and is detachably coupled at an outer peripheral portion of the hand piece main section (2) by connector liking with the mounting cylinder (55), and which captures an image signal of a target location picked up by the camera unit (31) from the linking connector part (56) through the connection wiring part and the cylindrical connector section (53), performs image processing on the captured image signal of the target location, and wirelessly transmits the same to destination apparatuses as target-location image information.

### TECHNICAL FIELD

The present invention relates to a wireless transmission unit for a dental instrument with a built-in camera, and a dental instrument with a built-in camera.

### BACKGROUND ART

In the field of dental treatment, there has recently been developed a dental instrument with a built-in camera, which also has an image picking-up function for intraoral observations in addition to a medical treatment or procedure function upon performing treatment or a procedure of an affected part (target location) in an oral cavity.

There has been proposed in, for example, Patent Document 1, a dental image pick-up apparatus configured in such a manner that a camera is attached to a dental instrument such as an ultrasonic scaler and connected to a controller for image processing by a cable, and camera image information of a target location image-processed by the controller is transmitted to an image display means by wireless to display an image of the target location in an oral cavity (Patent Document 1: FIG. 21, etc.).

In the case of this dental image pick-up apparatus, however, it is configured such that the camera image is transmitted to the image display means by wireless after the camera image is transmitted to the controller once by the cable. It is a current status that inclusive of this Patent Document 1, there are not yet such a wireless transmission unit that is detachably mounted to a dental instrument itself and transmits camera images to various destination apparatuses by wireless without interposing a controller to enable provision for an image display, and further such a dental instrument that is equipped with such a wireless transmission unit integrally and transmits camera images to various destination apparatuses by wireless to enable provision for an image display.

### CITATION LIST

### Patent Documents

Patent Document 1: Japanese Patent Application Laid-Open No. 2010-104652

### SUMMARY OF THE INVENTION

### Problems to be Solved by the Invention

A problem to be solved by the present invention resides in that there no exist such a wireless transmission unit for a dental instrument with a built-in camera and a dental instrument with a built-in camera that the wireless transmission unit is detachably mounted to the dental instrument with a built-in camera itself and transmits a camera image to various destination apparatuses by wireless to enable provision for an image display and that is capable of achieving an improvement in visibility of an affected part while improving the operability for a dentist or the like at the time of treatment or a procedure, and the promotion of patient understanding of treatment contents.

### Means for Solving the Problems

The present invention provides the most main feature in that there is provided a wireless transmission unit for use in a dental instrument for a built-in camera, in which a tool for performing treatment or a procedure of a target location in an oral cavity is provided on a distal end side of a housing, the camera for picking up the target location is built in the housing, and a power cable for supplying a required voltage is connected thereto, and which includes a mounting cylinder having a cylindrical connector section which is fitted onto an outer periphery of the housing and in which the other end of a connection wiring part having one end connected to the camera, is led outside the housing so that the cylindrical connector section is connected to the other end; and image processing wireless transmission means which has a linking connector part linked to the cylindrical connector section of the mounting cylinder and which is detachably coupled at an outer peripheral portion of the housing by connector linking with the mounting cylinder and captures an image signal of the target location picked up by the camera from the linking connector part through the connection wiring part and the cylindrical connector section, and which performs image processing on the captured image signal of the target location and wirelessly transmits the same to destination apparatuses as target-location image formation.

### Advantageous Effects of the Invention

According to the invention described in claim 1, since a wireless transmission unit is detachably mounted to a dental instrument with a built-in camera itself, and the wireless transmission unit captures an image signal of a target location picked up by the camera at the time of treatment or a procedure of an affected part, performs image processing thereon and wirelessly transmits the same to various destination apparatuses as target-location image information by wireless so as to be provided for an image display, it is possible to provide a wireless transmission unit used in a dental instrument with a built-in camera, which is capable of achieving an improvement in visibility of an affected part while improving operability for a dentist or the like at the time of treatment or a procedure of a target location, and further the promotion of patient understanding of the contents of treatment.

According to the invention described in claim 2, since a wireless transmission unit is detachably mounted to a dental instrument with a built-in camera itself, and at the time of treatment or a procedure of an affected part, image processing wireless transmission means of the wireless transmission unit supplies a drive voltage to the camera through a linking connector part, a cylindrical connector section of a mounting cylinder, and a connection wiring part and captures an image signal of a target location picked up by the camera from the linking connector part through the connecting wiring part and the cylindrical connector section, and performs image processing on the captured image signal of the target location, and wirelessly transmits the same to destination apparatuses as target-location image information so as to be provided for an image display, it is possible to provide a wireless transmission unit used in a dental instrument with a built-in camera, which is capable of achieving an improvement in visibility of an affected part while improving operability for a dentist or the like at the time of treatment or a procedure of a target location, and further the promotion of patient understanding of the contents of treatment as with the invention described in claim 1.

According to the invention described in claim 3, since a wireless transmission unit is detachably mounted to a dental instrument with a built-in camera itself, and at the time of treatment or a procedure of an affected part, image processing wireless transmission means of the wireless transmission unit supplies a drive voltage to the camera through a linking connector part, a cylindrical connector section of a mounting cylinder, and a connection wiring part and captures an image signal of a target location picked up by the camera from the linking connector part to a color image generating unit through the connecting wiring part and the cylindrical connector section to generate target-location color image information, and wirelessly transmits the target-location color image information from the transmitting transmitter to destination apparatuses so as to be provided for an image display, it is possible to provide a wireless transmission unit used in a dental instrument with a built-in camera, which is capable of achieving an improvement in visibility of an affected part while improving operability for a dentist or the like at the time of treatment or a procedure of a target location, and further the promotion of patient understanding of the contents of treatment as with the invention described in claim 1.

According to the invention described in claim 4, in the invention described in any of claims 1 to 3, the arrangement of mounting of the wireless transmission unit to the housing of the dental instrument with the built-in camera is such that when an assumed reference line extending in a projecting direction of the tool from the housing is assumed, the wireless transmission unit is arranged to be inclined by a predetermined angle to the right in the case of right-handed specifications and to the left in the case of left-handed specifications with respect to the assumed reference line as viewed from the distal end side of the housing. It is therefore possible to provide a wireless transmission unit used in a dental instrument with a built-in camera, which when performing treatment or a procedure of an affected part by using the dental instrument with the built-in camera, brings the root portion of the index finger into a state of not contacting (interfering in) the wireless transmission unit, and which is also capable of clearly visually recognizing the affected part and the distal end of the tool and is capable of ensuring good operability of the dental instrument with the built-in camera.

According to the invention described in claim 5, in the invention described in any of claims 1 to 4, each of the destination apparatuses is configured to be selected from a dental care unit, cart type image display means, a personal computer or a head mounted display, etc. which respectively have a receiving transmitter. It is therefore possible to provide a wireless transmission unit used in a dental instrument with a built-in camera, which is capable of reducing a dead angle area due to visual recognition of each part in an oral cavity, e.g., a root canal opening, a pulp horn, a pulpal floor, a cavity shadow, etc. while improving treatment operability or procedure operability for a dentist or the like being an operator, and of realizing information sharing about the target-location image information in plural persons concerned such as a dentist, a dental technician, etc. when the destination apparatus is the personal computer, and further of realizing the promotion of patient understanding of the treatment contents, for example when the destination apparatus is the head mounted display.

According to the invention described in claim 6, in the invention described in any of claims 1 to 5, the dental instrument with the built-in camera is configured to be selected from an air turbine hand piece, a micromotor hand piece, a syringe or a scaler, etc., which respectively have incorporated a camera therein. It is therefore possible to provide a wireless transmission unit used in a dental instrument with a built-in camera, which, when the treatment or procedure of the target location is carried out using these various dental instruments with a built-in camera, enables them to exert the above-described effects, respectively.

According to the invention described in claim 7, under the configuration that an outer peripheral cylindrical body configuring a wireless transmission unit is integrally formed with a dental instrument with a built-in camera itself, the wireless transmission unit captures an image signal of a target location picked up by the camera at the time of treatment or a procedure of an affected part and performs image processing thereon, and wirelessly transmits the same to various destination apparatuses by wireless as target-location image information so as to be provided for an image display. It is therefore possible to provide a dental instrument with a built-in camera, which is capable of achieving an improvement in visibility of an affected part while improving operability for a dentist or the like at the time of treatment or a procedure of a target location, and further the promotion of patient understanding of the contents of treatment.

According to the invention described in claim 8, under the configuration that an outer peripheral cylindrical body configuring a wireless transmission unit is integrally formed with a dental instrument with a built-in camera itself, upon treatment or a procedure of an affected part, image processing wireless transmission means of the wireless transmission unit supplies a drive voltage to the built-in camera through a linking connector part, a cylindrical connector section of a mounting cylinder, and a connection wiring part and captures an image signal of a target location picked up by the camera from the linking connector part to a color image generating unit through the connecting wiring part and the cylindrical connector section to generate target-location color image information, and wirelessly transmits the target-location color image information from the transmitting transmitter to destination apparatuses so as to be provided for an image display. It is therefore possible to provide a dental instrument with a built-in camera, which is capable of achieving an improvement in visibility of an affected part while improving operability for a dentist or the like at the time of treatment or a procedure of a target location, and further the promotion of patient understanding of the contents of treatment as with the invention described in claim 7.

According to the invention described in claim 9, in the invention described in claim 7 or 8, there can be provided a dental instrument with a built-in camera, which when performing treatment or a procedure of an affected part by using the dental instrument with a built-in camera with the arrangement of the outer peripheral cylindrical body configuring the wireless transmission unit as the right- or left-handed angular arrangement similar to the case of the invention described in claim 4, brings the root portion of the index finger into a state of not contacting (interfering in) the wireless transmission unit, and which is also capable of clearly visually recognizing the affected part and the distal end of the tool and is capable of ensuring good operability.

According to the invention described in claim 10, in the invention described in any of claims 7 to 9, there can be provided a dental instrument with a built-in camera, which brings about effects similar to the case of the invention described in claim 5 with a configuration that each of the destination apparatuses is selected from a dental care unit, cart type image display means, a personal computer or a head mounted display, etc. which respectively have a receiving transmitter.

According to the invention described in claim 10, in the invention described in any of claims 7 to 9, when the dental instrument with the built-in camera is selected from an air turbine hand piece, a micromotor hand piece, a syringe or a scaler, etc., which respectively have incorporated a camera therein, and these various dental instruments with a built-in camera are used to perform treatment or a procedure of a target location, they can exert the above-described effects respectively.

### BRIEF DESCRIPTION OF THE DRAWINGS

FIG. 1 is a schematic front view illustrating one example of a dental instrument with a built-in camera, which is equipped with a wireless transmission unit according to an embodiment 1 of the present invention;

FIG. 2 is a schematic exploded perspective view illustrating one example of the dental instrument with the built-in camera, which is equipped with the wireless transmission unit according to the present embodiment 1;

FIG. 3 is a schematic system diagram of the wireless transmission unit and the dental instrument with the built-in camera according to the present embodiment 1;

FIG. 4 is a configuration block diagram of an image processing wireless transmission means of the wireless transmission unit according to the present embodiment 1;

FIG. 5 is a schematic configuration diagram of a camera unit built in the dental instrument in the present embodiment 1;

FIG. 6 is a side view of the cameral unit built in the dental instrument in the present embodiment 1;

FIG. 7 is a sectional view taken along line A-A of FIG. 5;

FIG. 8 is an explanatory diagram illustrating destination apparatuses of target-location image information wirelessly transmitted by the wireless transmission unit according to the present embodiment 1;

FIG. 9 is a diagram illustrating a screen display example of target-location color image information wirelessly transmitted by the wireless transmission unit according to the present embodiment 1;

FIG. 10 is a schematic front view illustrating another example of a dental instrument with a built-in camera, which is equipped with a wireless transmission unit according to an embodiment 2 of the present invention;

FIG. 11 is a schematic front view illustrating a further example of a dental instrument with a built-in camera, which is equipped with a wireless transmission unit according to an embodiment 3 of the present invention;

FIG. 12 is a schematic explanatory view illustrating the arrangement of mounting of the wireless transmission unit in the dental instrument with the built-in camera equipped with the wireless transmission unit according to the present embodiment 1 to a housing (hand piece main section);

FIG. 13 is a schematic perspective view illustrating a use mode of the dental instrument with the built-in camera, which is equipped with the wireless transmission unit shown in FIG. 12; and

FIG. 14 is a schematic exploded perspective view illustrating a dental instrument with a built-in camera, which is equipped with a wireless transmission unit according to an embodiment 4 of the present invention.

### MODE FOR CARRYING OUT THE INVENTION

An object of providing by the present invention, a wireless transmission unit for use in a dental instrument with a built-in camera, which is capable of wirelessly transmitting camera images to various destination apparatuses and achieving an improvement in visibility of an affected part while improving operability for a dentist or the like during treatment or a procedure, and the promotion of patient understanding of the contents of treatment is realized by a configuration of a wireless transmission unit for use in a dental instrument with a built-in camera, in which a tool for performing treatment or a procedure of a target location in an oral cavity is provided on a distal end side of a housing, the camera for picking up the target location is built in the housing, and a power cable for supplying a required voltage is connected thereto; which includes a mounting cylinder having a cylindrical connector section which is fitted onto an outer periphery of the housing and in which the other end of a connection wiring part having one end connected to the camera, is led outside the housing so that the cylindrical, connector section is connected to the other end, image processing wireless transmission means which has a linking connector part linked to the cylindrical connector section of the mounting cylinder, and a power supply connector part and which is detachably coupled at an outer peripheral portion of the housing by connector linking with the mounting cylinder, and a power supply connector unit which is connected to the power cable within the housing and connected to an end of a power auxiliary cable led outside the housing so as to be linked to the power supply connector part thereby to supply a required voltage to the image processing wireless transmission means; and in which the image processing wireless transmission means supplies a drive voltage to the camera through the linking connector part, the cylindrical connector section and the connection wiring part and captures an image signal of the target location picked up by the camera from the cylindrical connector section through the connecting wiring part and the cylindrical connector section, and performs image processing on the captured image signal of the target location and wirelessly transmits the same to destination apparatuses as target-location image information.

### EMBODIMENTS

A wireless transmission unit for a dental instrument with a built-in camera according to each embodiment of the present invention, and a dental instrument with a built-in camera will be described below in detail with reference to the drawings.

### Embodiment 1

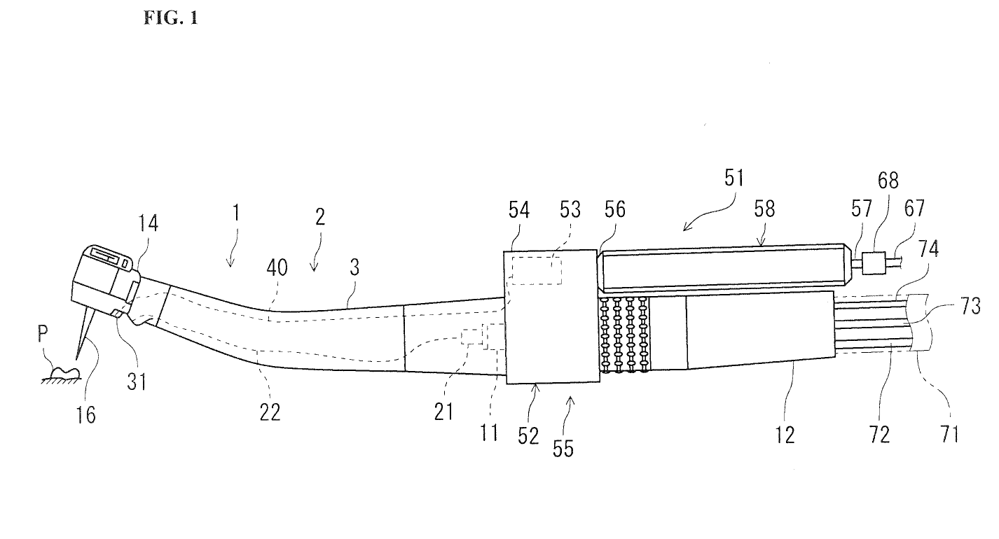

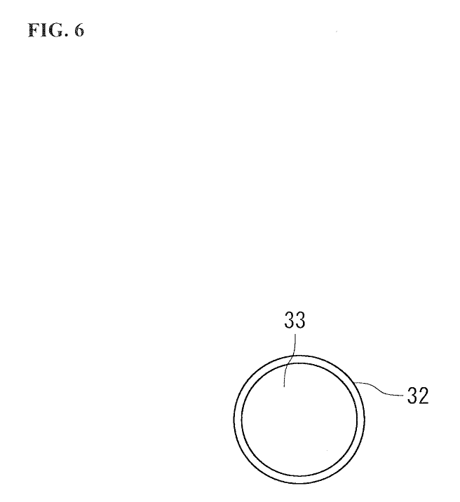

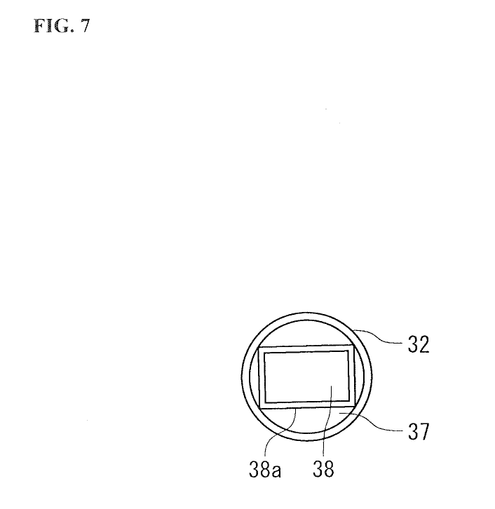

A wireless transmission unit 51 for a dental instrument with a built-in camera according to the present embodiment 1 will be described with reference to FIGS. 1 through 8.

The present embodiment 1 is intended to be applied to an air turbine-driven camera built-in hand piece 1 as one of dental instrument with a built-in camera.

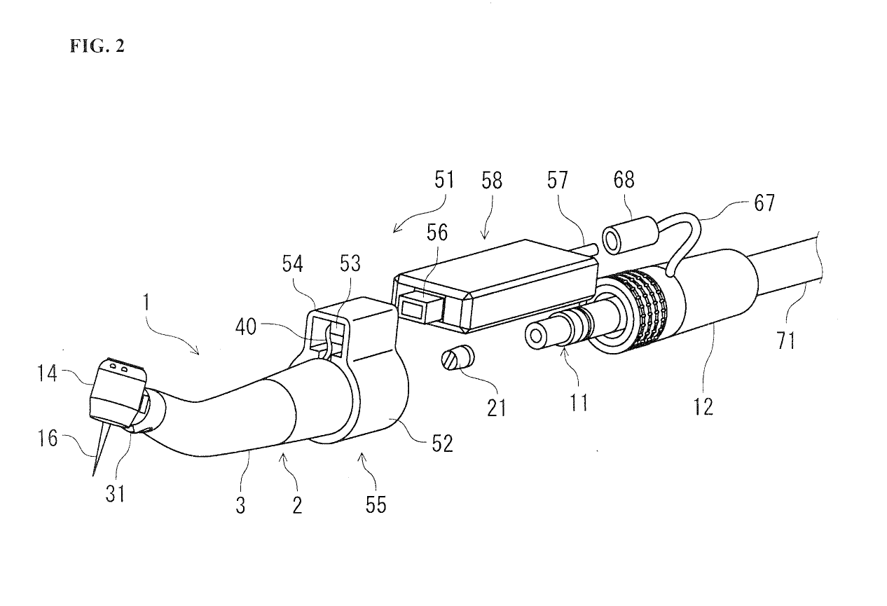

The camera built-in hand piece 1 of the present embodiment 1 has, as illustrated in FIGS. 1 and 2, a grip part 3 which configures a hand piece main section 2 provided on its tip side with a head part 14 for detachably mounting a cutting tool 16 for the treatment of an affected part P of a tooth which is a target location in an oral cavity, and a dental hose section 12 provided with a coupling portion 11 detachably mounted within the hand piece main section 2. A housing is configured by the grip part 3 and the dental hose section 12 provided with the coupling portion 11. The dental hose section 12 is provided with a connecting hose 71 connected to an unillustrated dental care unit. Incidentally, although the grip part 3 side is also provided with a coupling portion corresponding to the coupling portion 11, its illustration will be omitted.

An air pipe 72 which constitutes an air flow path system for guiding compressed air supplied from the unillustrated dental care unit, and a water pipe 73 which constitutes a water flow path system for guiding pressurized water and a power cable 74 for supplying a required voltage are installed inside the connecting hose 71.

Then, there are provided within the dental hose section 12, the coupling portion 11, and the grip part 3 including the head part 14, although omitted in detailed structure, an air flow path system for guiding an air flow for an air turbine force-fed to the head part 14 of the hand peace main section 2 via the dental hose section 12 and guiding compressed air for an air flow injected toward the cutting tool 16 from the head part 14, and a water flow path system for guiding pressurized water for a water flow injected toward the cutting tool 16 from the head part 14, which is force-fed to the head part 14 of the hand peace main section 2 via the dental hose section 12.

Further, a light source part 21 comprised of, for example, an LED (Light Emitting Diode) element (one having a drive voltage DC 3.3V, for example) for illumination is arranged at the tip portion of the coupling portion 11, which occupies the inside of the grip part 3. Furthermore, a rod fiber 22 is placed in such an arrangement that one end thereof is exposed at a position near the head part 14 in the grip part 3 and the other end thereof is faced to the vicinity of the light source part 21. Light emitted from the light source part 21 is configured to be guided via the rod fiber 22 and emitted from one end thereof to a region of a target location (affected part P) by the cutting tool 16 as illumination light.

The light source part 21 is lit and driven by a driving voltage supplied from the power cable 74 although its wiring system is not shown in the drawing.

Further, a camera unit 31 which configures a camera for picking up the target location is built in at the position closer to the emission end of the rod fiber 22, of the head part 14 in the grip part 3.

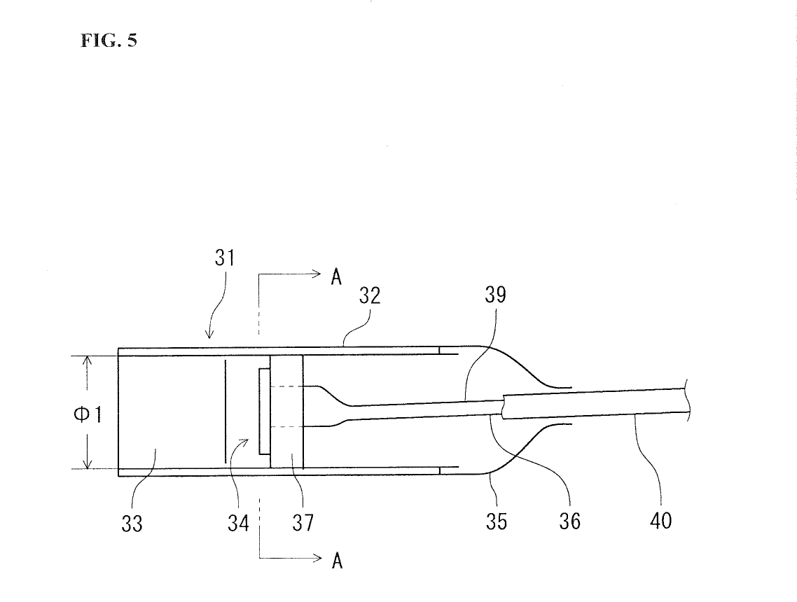

Here, the camera unit 31 will be described in detail with reference to FIGS. 5 through 7.

The camera unit 31 has, for example, a cylindrical support tube 32 which is 1.2 mm in diameter, about 1.1 mm in inner diameter, and 3 mm in length, a condenser lens unit 33 having a diameter of Φ1=1.1 mm, which is placed to face a light entrance end thereof to one end face of this support tube 32, an image pick-up unit 34 disposed opposite to the condenser lens unit 33 with a predetermined interval spaced therebetween within the support tube 32, and a cover member 35 fitted to the range from the other end face side of the support tube 32 to within the support tube 32.

The image pick-up unit 34 is equipped with a disk-shaped support substrate 37 having a diameter of 1.1 mm securely fixed in a state in which the center thereof is aligned with the optical axis of the condenser lens unit 33 within the support tube 32, and a color image sensor (CMOS: Complementary Metal Oxide Semiconductor) 38 mounted in a state in which color pixels are arranged on the surface of a sensor substrate 38a having outer dimensions of 0.84×0.74 mm and a thickness of 0.1 mm so that the number of the pixels assumes 320×240 pixels, and the center thereof coincides with the optical axis of the condenser lens unit 33.

The condenser lens unit 33 adopts, for example, one having optical characteristics of a viewing angle of 70 degrees and a focus range 3-50 mm.

Further, the camera unit 31 is configured to connect a signal cable 36 for transmitting an image signal of a picked-up target location and a drive cable 39 for supplying a voltage for the operation of a color image sensor 38 to the color image sensor 38, internally install these in a camera cable 40 as a connection wiring part, and allow the camera cable 40 to penetrate the cover member 35 so as to lead out to the rear side.

Next, the wireless transmission unit 51 according to the present embodiment 1 will be described in detail with reference to FIGS. 1 through 4.

As illustrated in FIGS. 1 and 2, the wireless transmission unit 51 according to the present embodiment 1 is provided at each end face with a mounting cylinder 55 having a mounting cylindrical part 52 fitted on the outer periphery of the grip part 3 on the side which is closer to the coupling portion 11, and a connector cylindrical unit 54 formed integrally with the mounting cylindrical part 52, which is provided with a cylindrical connector section 53 connected to the other end of the camera cable 40 having one end connected to the camera unit 31 and configuring the connection wiring part which leads out a penetration portion to the outside of the grip part 3 via the inside thereof as waterproof specifications; a linking connector part 56 coupled to the cylindrical connector section 53 of the mounting cylinder 55; and a power supply connector part 57. The wireless transmission unit 51 has an image processing wireless transmission means 58 having an external appearance formed in a substantially rectangular parallelepiped shape, which is coupled detachably at the outer peripheral portion of the grip part 3 by connector linking with the mounting cylinder 55, and a power supply connector unit 68 which is connected to the power cable 74 within the dental hose section 12, e.g., which is connected to the end of a power auxiliary cable 67 led out to the outside from a joint portion between the coupling portion 11 and the dental hose section 12 and connected to the power supply connector part 57 to thereby supply a required voltage to the image processing wireless transmission means 58.

The penetration portion of the grip part 3 in the connection wiring part can be brought to resistant autoclave specifications by being taken as the waterproof specifications.

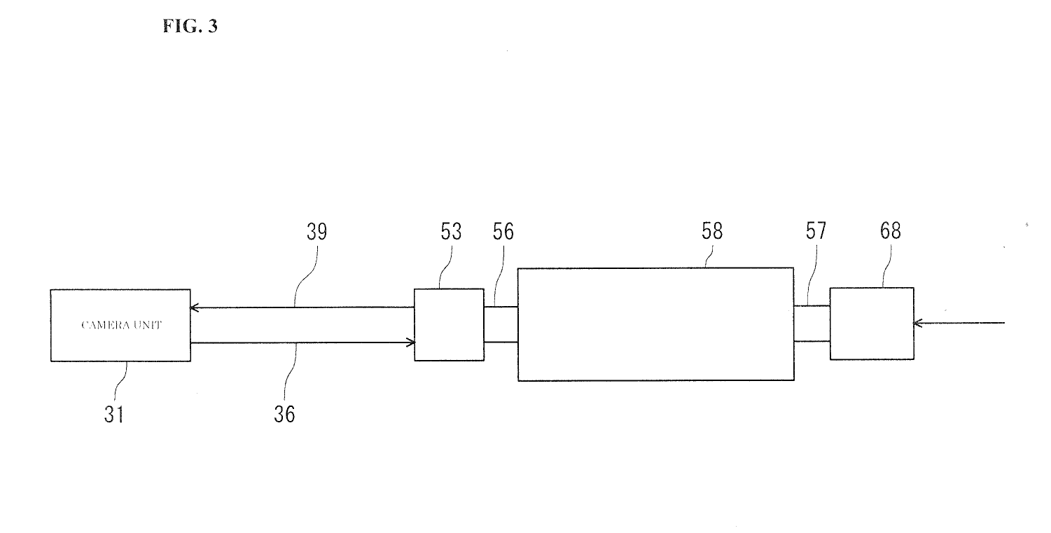

A connection system of the wireless transmission unit 51 and the camera unit 31 is schematically illustrated in FIG. 3.

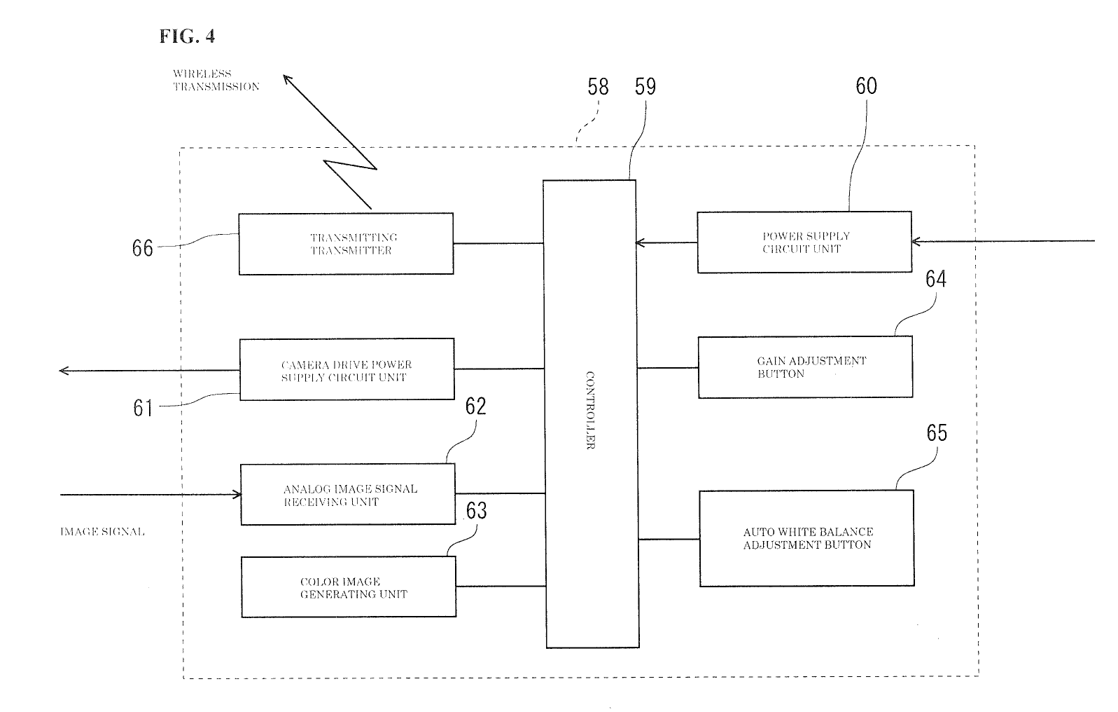

Next, the configuration contents of the image processing wireless transmission means 58 will be described with reference to FIG. 4.

The image processing wireless transmission means 58 has a controller 59 which performs control of the image processing wireless transmission means 58, a power supply circuit unit 60 which fetches a required voltage supplied via the power supply connector part 57 and performs the supply of power necessary for each part of the image processing wireless transmission means 58, a camera drive power supply circuit unit 61 for driving the camera unit 31, an analog image signal receiving unit 62 which receives an image signal of a target location picked up by the camera unit 31, a color image generating unit 63 which generates target-location color image information, based on the image signal, a gain adjustment button 64 which adjusts the gain at the time of image generation processing, an auto white balance adjustment button 65 which adjusts the white balance at the time of the image generation processing, and a transmitting transmitter 66 which wirelessly transmits the purified target-location color image information to each destination apparatus.

As the wireless communication system of the transmitting transmitter 66, there may be mentioned examples of, e.g., an IEEE802.11-based wireless LAN system and a wireless communication system by infrared communication.

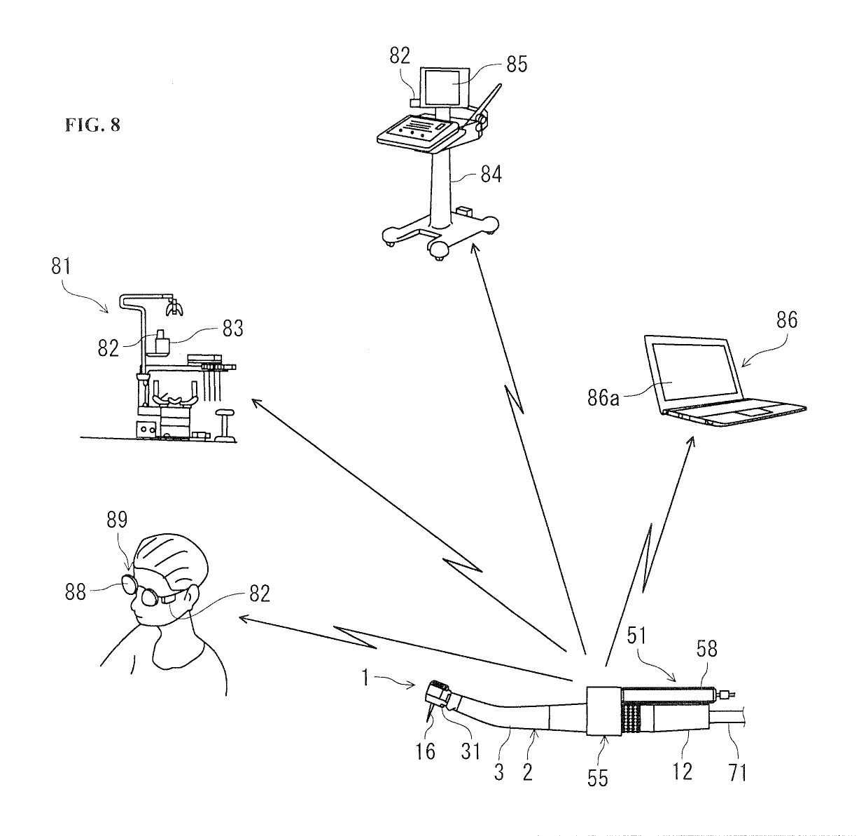

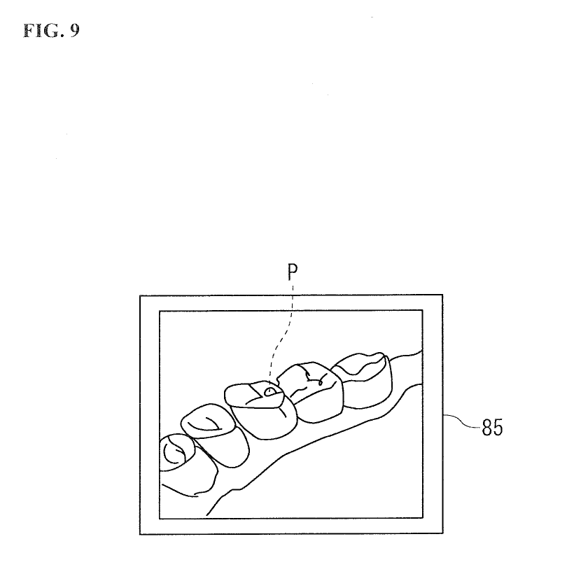

Next, the operation of the wireless transmission unit 51 according to the present embodiment 1, which is equipped with the camera built-in hand piece 1, will be described with reference to FIGS. 8 and 9 while principally taking the operation for the transmission of the target-location color image information from the wireless transmission unit 51 to each destination apparatus.

In the camera built-in hand piece 1 of the present embodiment 1, when the cutting tool 16 mounted to the head part 14 is rotationally driven to perform treatment of an affected part P of a tooth corresponding to a target location of a certain patient, a drive voltage is supplied from the camera drive power supply circuit unit 61 of the image processing wireless transmission means 58 to the camera unit 31 through the linking connector part 56, the cylindrical connector section 53, and the connection wiring part, so that the image pick-up unit 34 is brought to an operating state to thereby pick up an image of the affected part P.

A signal of the image of the affected part P picked up by the image pick-up unit 34 is transmitted from the linking connector part 56 of the image processing wireless transmission means 58 to the analog image signal receiving unit 62 through the connection wiring part and the cylindrical connector section 53. The color image generating unit 63 generates target-location color image information on the basis of the image signal of the affected part P received by the analog image signal receiving unit 62.

The target-location color image information generated by the color image generating unit 63 is wirelessly transmitted from the transmitting transmitter 66 to such various destination apparatuses as illustrated in FIG. 8.

As the destination apparatuses in this case, there may be mentioned, as shown in FIG. 8, e.g., examples such as an image display means 83 equipped with a receiving transmitter 82 mounted to a dental care unit 81 in a dental clinic, which is set to the same wireless communication standard specifications as the transmitting transmitter 66, a cart type image display means 85 equipped with a receiving transmitter 82 placed in a cart 84 in the dental clinic, which is set to the same wireless communication standard specifications as the transmitting transmitter 66, a personal computer 86 equipped with a receiving transmitter 82 set to the same wireless communication standard specifications as the transmitting transmitter 66 in the dental clinic, a head mounted display 89 (for example, spectacle type) with a receiving transmitter 82 and an image display means 88 set to the same wireless communication standard specifications as the transmitting transmitter 66 being mounted to a head-mounted head holder 87 prepared in advance in the dental clinic, etc.

Thus, for example, an affected part P and target-location color image information of its peripheral portion are displayed on the screen of the cart type image display means 85 as illustrated in FIG. 9.

The image display means 83 of the dental care unit 81, a display unit 86a of the personal computer 86, and the image display means 88 of the head mounted display 89 are also similar to the case of the cart type image display means 85.

In the case of the above-described cart type image display means 85, if the cart 84 is placed closer to an operator actually carrying out the treatment of the affected part P, the operator is able to visually confirm the image display means 85 and clearly recognize the affected part P and the target-location color image information of its peripheral portion without using a mirror (Dental Mirror). It is therefore possible to reduce a dead angle area by the visual recognition of each part in an oral cavity, e.g., a root canal opening, a pulp horn, a pulpal floor, a cavity shadow, etc. and thereby achieve a stress reduction at the time of treatment.

Even in the case of the dental care unit 81, since the operator is able to visually confirm the image display means 83 and clearly recognize the affected part P and the target-location color image information of its peripheral portion without using the mirror, it is possible to reduce a dead angle area by the visual recognition of each part in the oral cavity, e.g., the root canal opening, pulp horn, pulpal floor, cavity shadow, etc. while enhancing operability in operation and achieve a reduction in stress at the time of treatment.

In the case of the personal computer 86, since the target-location color image information can be transmitted to any other number of personal computers (not shown) constructed with a LAN connection in advance in the dental clinic, it is also extremely simple and easy to realize information sharing about the target-location color image information in plural persons concerned such as a dentist, a dental technician, etc.

In the case of the head mounted display 89, since any person is able to mount the head mounted display 89 on the head thereof, the promotion of patient understanding to the treatment contents can also be achieved by causing the person to visually recognize target-location color image information through the image display means 88 in a state in which the head mounted display 89 is mounted on, for example, the head of the patient and the treatment is actually being performed.

### Embodiment 2

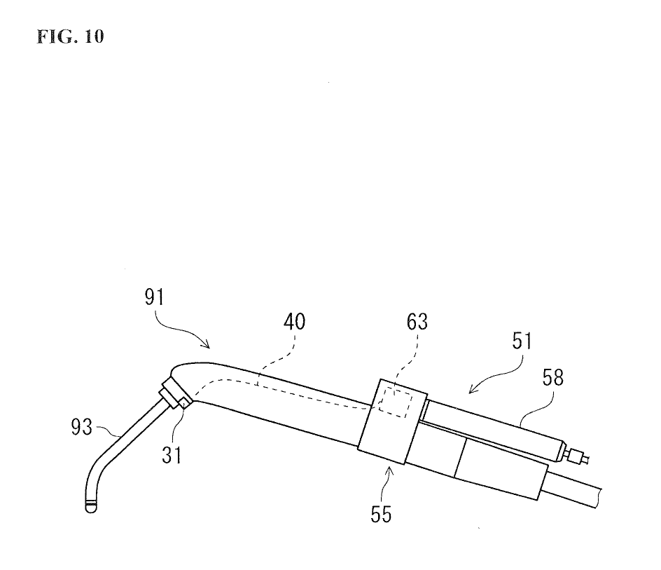

An embodiment 2 of the present invention will be described with reference to FIG. 10.

The present embodiment 2 is configured to mount a wireless transmission unit 51 to a camera built-in syringe (camera built-in multiway syringe) 91 being a dental instrument with a camera unit 31 built therein on the basis of a configuration similar to the above-described case.

A syringe body 92 which configures the camera built-in syringe 91 is equipped with a conduit of pressurized fluid (water or chemical solution), a conduit of compressed air, and a control valve for controlling those conduits respectively as is publicly known. The syringe body 92 is configured to selectively inject a plurality of types of dental working fluid from an injection nozzle 93 to the affected part P depending on the dental treatment and treat the affected part P.

Then, the camera unit 31, the connection wiring part, and further the power cable are incorporated into a position near a mounting portion of the injection nozzle 93 in the syringe body 92 on the basis of a configuration similar to the case of the embodiment 1 although omitted in detailed description to thereby configure a camera built-in syringe 91. Further, the wireless transmission unit 51 similar to the case of the embodiment 1 is configured to be mounted to the syringe body 92.

Even by the present embodiment 2, when the treatment of the affected part P is carried out by the camera built-in syringe 91, target-location color image information is wirelessly transmitted from a transmitting transmitter 66 of the wireless transmission unit 51 to such various destination apparatuses as illustrated in FIG. 8 to enable provision for an image display. Thus, as with the case of the embodiment 1, it is possible to achieve a reduction in dead angle area by visual recognition of each part in an oral cavity, e.g., a root canal opening, a pulp horn, a pulpal floor, a cavity shadow, etc. while improving treatment operability on the operator side, and further the promotion of understanding to the contents of treatment on the patient side.

### Embodiment 3

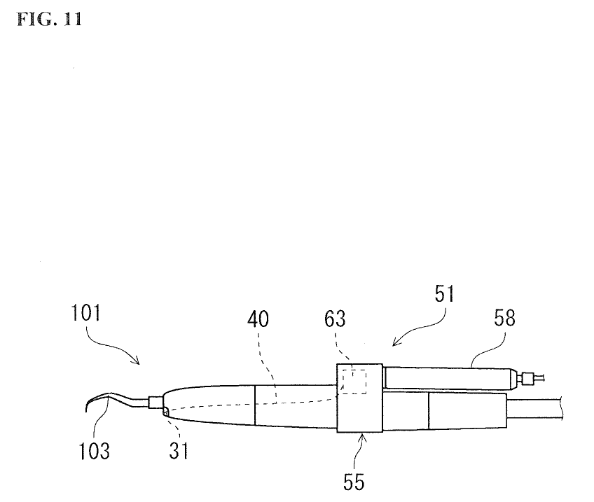

An embodiment 3 of the present invention will be described with reference to FIG. 11.

The present embodiment 3 is configured to mount a wireless transmission unit 51 to a camera built-in scaler 101 being a dental instrument having incorporated a camera unit 31 therein, based on a configuration similar to the above-described case.

A scaler body 102 which configures the camera built-in scaler 101 is, although omitted in detailed description, for example, one in which an ultrasonic vibrator with an ultrasonic vibration source (piezoelectric element) and a vibration transmitting member (horn) formed integrally is placed in the scaler body 102, a dental tip 103 which effectively acts on an affected part P is arranged in the tip of the scaler body 102, and the dental tip 103 is driven by ultrasonic vibrations to thereby perform scaling (tartar removal) of tooth surfaces, between the teeth, and below gingival edges, etc. in an oral cavity.

Then, the camera unit 31 and the connection wiring part are built in a position near an attachment portion of the dental tip 103 in the scaler body 102, based on a configuration similar to the case of the embodiment 1. Further, the wireless transmission unit 51 similar to the case of the embodiment 1 is configured to be mounted to the scaler body 102.

Even by the present embodiment 3, when the treatment of the affected part P is carried out by the camera built-in scaler 101, target-location color image information is wirelessly transmitted from a transmitting transmitter 66 of the wireless transmission unit 51 to such various destination apparatuses as illustrated in FIG. 8 to enable provision for an image display. Thus, as with the case of the embodiment 1, it is possible to achieve a reduction in dead angle area by visual recognition of each part in an oral cavity, e.g., a root canal opening, a pulp horn, a pulpal floor, a cavity shadow, etc. while improving treatment operability on the operator side, and further the promotion of understanding to the contents of treatment on the patient side.

Each of the configurations of the embodiments 1 through 3 described above is applicable even to various existing dental instruments in a simple manner.

Incidentally, it is also possible to make applications as a configuration in which the camera unit 31 is externally attached to a syringe body 92 in place of the camera built-in syringe 91 and the camera built-in scaler 101 and the wireless transmission unit 51 is mounted to the syringe body 92, and a configuration in which the camera unit 31 is externally attached to the scaler body 102 and the wireless transmission unit 51 is mounted to the scaler body 102.

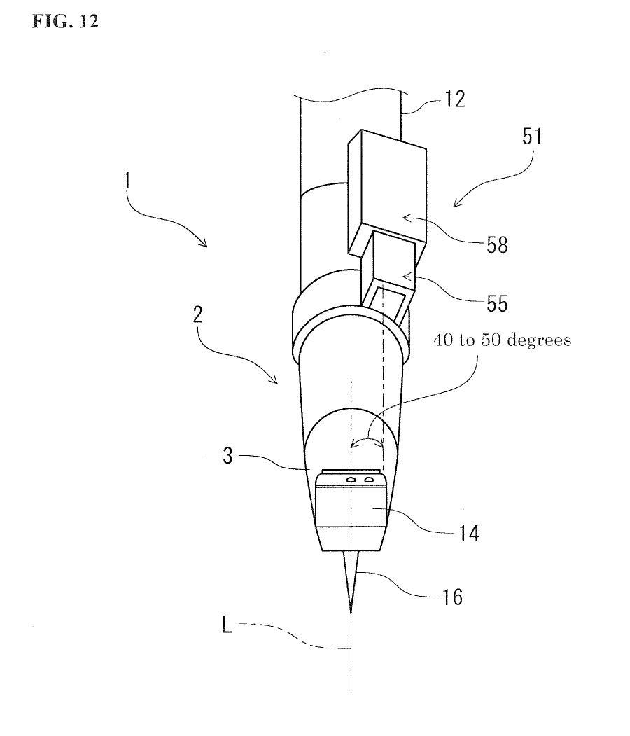

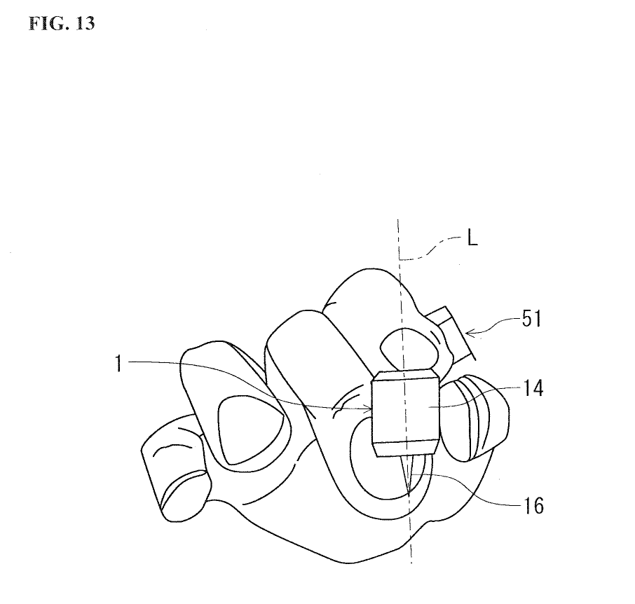

FIGS. 12 and 13 respectively schematically illustrate a preferred example of a mounting arrangement of the wireless transmission unit 51 to the hand piece main section 2 in the camera built-in hand piece 1 of the embodiment 1 described above.

That is, when an assumed reference line L extending in a projecting direction of the cutting tool 16 from the grip part 3 is assumed, the wireless transmission unit 51 is arranged to be inclined by a predetermined angle (e.g., 40 to 50 degrees) to the right with respect to the assumed reference line L as viewed from the tip side of the hand piece main section 2 in the case of right-handed specifications. Further, in the case of left-handed specifications, although not shown in the drawing, the wireless transmission unit 51 is arranged to be inclined by a predetermined angle (e.g., 40 to 50 degrees) to the left with respect to the assumed reference line L as viewed from the tip side of the hand piece main section 2.

The following effects are brought about by adopting such arrangements of the wireless transmission unit 51.

That is, when the treatment of the affected part P is performed using the camera built-in hand piece 1, the operator turns the hand piece main section 2 to take an appropriate angle using the index finger and thumb while supporting the hand piece main section 2 with the middle finger to thereby provide an optimal position for the affected part P.

In this case, as illustrated in FIG. 13, the upper surface of the head part 14 of the hand piece main section 2, and the abdominal portion of the index finger of the operator are preferably made flush with each other. This is applied similarly even to the case of the left-handed specifications.

At this time, as illustrated schematically in FIG. 13, the root portion of the index finger becomes a state of being in no contact with (not interfering in) the wireless transmission unit 51 by the above-described arrangement of the wireless transmission unit 51 in the case of the right-handed specifications, so that good operability of the camera built-in hand piece 1 can be ensured. Although not illustrated even in the case of the left-handed specifications, it is possible to ensure good operability of the camera built-in hand piece 1 in like manner.

Further, by the above-described arrangement of the wireless transmission unit 51, it also becomes possible for the operator to clearly visually recognize the affected part P and the distal end of the cutting tool 16 without being blocked by the head part 14. Even from this point, good operability of the camera built-in hand piece 1 can be ensured.

The configuration related to the mounting arrangement of the wireless transmission unit 51 with respect to the hand piece main section 2 in such a camera built-in hand piece 1 as illustrated in FIGS. 12 and 13 is similarly applicable even to the camera built-in syringe 91 with the camera unit 31 of the embodiment 2 built therein, and the camera built-in scaler 101 of the embodiment 3, respectively, and similar effects can be exhibited.

### Embodiment 4

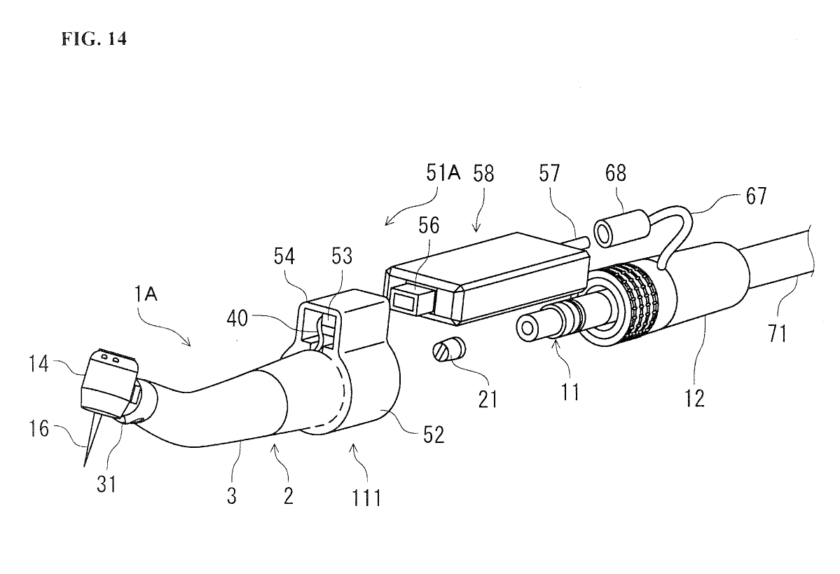

A dental instrument with a built-in camera according to an embodiment 4 of the present invention will be described with reference FIG. 14.

The present embodiment 4 is one applied to an air turbine-driven camera built-in hand piece 1A as one of dental instruments with a built-in camera as with the case of the embodiment 1.

In the camera built-in hand piece 1A of the present embodiment 4, the same elements as in the case of the camera built-in hand piece 1 illustrated in FIG. 1 are denoted by the same reference numerals, and their detailed description will be omitted.

Although the camera built-in hand piece 1A of the present embodiment 4 is substantially similar to the case of the camera built-in hand piece 1 illustrated in FIG. 1 in basic configuration, as illustrated in FIG. 14, the camera built-in hand piece 1A is configured to integrally form an outer peripheral cylindrical body 111 with the grip part 3 in place of the mounting cylinder 55 at the outer periphery of the grip part 3 on the side closer to the coupling portion 11 and to provide a connector cylindrical unit 54 and a cylindrical connection section 53 similar to the already-described case in the outer peripheral cylindrical body 111, and is characterized by combining the outer peripheral cylindrical body 111, the image processing wireless transmission means 58 equipped with the linking connector part 56 and the power supply connector part 57, the power supply connector unit 68, and the power auxiliary cable 67 into a wireless transmission unit 51A.

The camera built-in hand piece 1A equipped with the wireless transmission unit 51A of the present embodiment 4 can also exhibit various operations and effects similar to the case of the camera built-in hand piece 1 equipped with the wireless transmission unit 51 of the already-described embodiment 1 on the basis of the configuration in which the grip part 3 and the outer peripheral cylindrical body 111 are integrally formed.

Incidentally, it is also possible to adopt a configuration in which the outer peripheral cylindrical body 111 is integrally formed on the outer periphery of a dental horse section 12.

Also, as the arrangement of the outer peripheral cylindrical body 111 configuring the wireless transmission unit 51A in the camera built-in hand piece 1A equipped with the wireless transmission unit 51A of the present embodiment 4, the outer peripheral cylindrical body 111 can also be arranged to be inclined by a predetermined angle (e.g., 40 to 50 degrees) to the right with respect to an assumed reference line L as viewed from the tip side of the hand piece main section 2 in the case of right-handed specifications or arranged to be inclined by a predetermined angle (e.g., 40 to 50 degrees) to the left with respect to the assumed reference line L as viewed from the tip side of the hand piece main section 2 in the case of left-handed specifications as with the case illustrated in FIG. 12. Thus, as with the case already described above, the operator is also able to clearly visually recognize an affected part P and the distal end of a cutting tool 16 without being blocked by a head part 14 of the camera built-in hand piece 1A and to secure good operability of the camera built-in hand piece 1A.

Further, the structure of the outer peripheral cylindrical body 111 in the wireless transmission unit 51A of the present embodiment 4 is similarly applicable even to the camera built-in syringe 91 with the camera unit 31 of the embodiment 2 built therein, and the camera built-in scaler 101 of the embodiment 3 respectively. Even in these cases, operations and effects similar to the already-described case can be exhibited.

### INDUSTRIAL APPLICABILITY

The present invention is widely applicable to various dental instruments such as an air turbine hand piece, a micromotor hand piece, a laser hand piece, a syringe or a scaler, etc., which respectively have incorporated a camera therein.

### DESCRIPTION OF REFERENCE NUMERALS

1 camera built-in hand piece
1A camera built-in hand piece
2 hand piece main section
3 grip part
11 coupling portion
12 dental hose section
14 head part
16 cutting tool
21 light source part
22 rod fiber
31 camera unit
32 support tube
33 condenser lens unit
34 image pick-up unit
35 cover member
36 signal cable
37 support substrate
38 color image sensor
38a sensor substrate
39 drive cable
40 camera cable
51 wireless transmission unit
51A wireless transmission unit
52 mounting cylindrical part
53 cylindrical connector section
54 connector cylindrical unit
55 mounting cylinder
56 linking connector part
57 power supply connector part
58 image processing wireless transmission means
59 controller
60 power supply circuit unit
61 camera drive power supply circuit unit
62 analog image signal receiving unit
63 color image generating unit
64 gain adjustment button
65 auto white balance adjustment button
66 transmitting transmitter
67 power auxiliary cable
68 power supply connector unit
71 connecting hose
72 air pipe
73 water pipe
74 power cable
81 dental care unit
82 receiving transmitter
83 image display means
84 cart
85 image display means
86 personal computer
86a display unit
87 head-mounted head holder
88 image display means
89 head mounted display
91 camera built-in syringe
92 syringe body
93 injection nozzle
101 camera built-in scaler
102 scaler body
103 dental tip
111 outer peripheral cylindrical body
P affected part
L assumed reference line

1. A wireless transmission unit for use in a dental instrument for a built-in camera, in which a tool for performing treatment or a procedure of a target location in an oral cavity is provided on a distal end side of a housing, the camera for picking up the target location is built in the housing, and a power cable for supplying a required voltage is connected thereto, said wireless transmission unit comprising:
a mounting cylinder having a cylindrical connector section which is fitted onto an outer periphery of the housing and in which the other end of a connection wiring part having one end connected to the camera, is led outside the housing to connect the cylindrical connector section to the other end; and
image processing wireless transmission means which has a linking connector part linked to the cylindrical connector section of the mounting cylinder and is detachably coupled at an outer peripheral portion of the housing by connector linking with the mounting cylinder, and which captures an image signal of the target location picked up by the camera from the linking connector part through the connection wiring part and the cylindrical connector section, and which performs image processing on the captured image signal of the target location and wirelessly transmits the same to destination apparatuses as target-location image formation.

2. A wireless transmission unit for use in a dental instrument with a built-in camera, in which a tool for performing treatment or a procedure of a target location in an oral cavity is provided on a distal end side of a housing, the camera for picking up the target location is built in the housing, and a power cable for supplying a required voltage is connected thereto, said wireless transmission unit comprising:
a mounting cylinder having a cylindrical connector section which is fitted onto an outer periphery of the housing and in which the other end of a connection wiring part having one end connected to the camera, is led outside the housing to connect the cylindrical connector section to the other end;
image processing wireless transmission means which has a linking connector part linked to the cylindrical connector section of the mounting cylinder, and a power supply connector part and which is detachably coupled at an outer peripheral portion of the housing by connector linking with the mounting cylinder; and
a power supply connector unit which is connected to the power cable within the housing and connected to an end of a power auxiliary cable led outside the housing so as to be linked to the power supply connector part thereby to supply a required voltage to the image processing wireless transmission means,
wherein the image processing wireless transmission means is configured to supply a drive voltage to the camera through the linking connector part, the cylindrical connector section and the connection wiring part and capture an image signal of the target location picked up by the camera from the linking connector part through the connecting wiring part and the cylindrical connector section and to perform image processing on the captured image signal of the target location and thereby wirelessly transmit the same to destination apparatuses as target-location image information.

3. A wireless transmission unit for use in a dental instrument with a built-in camera, in which a tool for performing treatment or a procedure of a target location in an oral cavity is provided on a distal end side of a housing, the camera for picking up the target location is built in the housing, and a power cable for supplying a required voltage is connected thereto, said wireless transmission unit comprising:
a mounting cylinder having a cylindrical connector section positioned on outer periphery of the housing and in which the other end of a connection wiring part having one end connected to the camera, is led outside the housing to connect the cylindrical connector section to the other end;
image processing wireless transmission means which has a linking connector part linked to the cylindrical connector section of the mounting cylinder, and a power supply connector part and is detachably coupled at an outer peripheral portion of the housing by connector linking with the mounting cylinder, and which is equipped with a camera power supply circuit unit for driving the camera, a color image generating unit which generates target-location color image information, based on the image signal of the target location picked up by the camera, and a transmitting transmitter which wirelessly transmits the target-location color image information; and
a power supply connector unit which is connected to the power cable within the housing and connected to an end of a power auxiliary cable led outside the housing so as to be linked to the power supply connector part thereby to supply a required voltage to the image processing wireless transmission means,
wherein a drive voltage is supplied from the camera power supply circuit unit of the image processing wireless transmission means to the camera through the linking connector part, the cylindrical connector section, and the connection wiring part, and an image signal of the target location picked up by the camera is captured from the linking connector part to the color image generating unit through the connection wiring part and the cylindrical connector section to generate target-location color image information, and the target-location color image information is wirelessly transmitted from the transmitting transmitter to destination apparatuses.

4. The wireless transmission unit for use in the dental instrument with the built-in camera according to claim 1, wherein the arrangement of mounting of the wireless transmission unit to the housing of the dental instrument with the built-in camera is such that when an assumed reference line extending in a projecting direction of the tool from the housing is assumed, the wireless transmission unit is arranged to be inclined by a predetermined angle to the right in the case of right-handed specifications and to the left in the case of left-handed specifications with respect to the assumed reference line as viewed from the distal end side of the housing.

5. The wireless transmission unit for use in the dental instrument with the built-in camera according to claim 1, wherein each of the destination apparatuses is selected from a dental care unit, cart type image display, a personal computer or a head mounted display which respectively have a receiving transmitter.

6. The wireless transmission unit for use in the dental instrument with the built-in camera according to claim 1, wherein the dental instrument with the built-in camera is selected from an air turbine hand piece, a micromotor hand piece, a syringe or a scaler which respectively have incorporated a camera therein.

7. A dental instrument with a built-in camera, comprising a tool for performing treatment or a procedure of a target location in an oral cavity provided on a distal end side of a housing, the camera for picking up the target location being built in the housing, and a power cable for supplying a required voltage connected to the housing, and
a wireless transmission unit according to claim 1,
wherein the mounting cylinder is configured to as an outer peripheral cylindrical body integrally formed on an outer periphery of the housing.

8. A dental instrument with a built-in camera, comprising a tool for performing treatment or a procedure of a target location in an oral cavity provided on a distal end side of a housing, the camera for picking up the target location being built in the housing, and a power cable for supplying a required voltage connected to the housing, and
a wireless transmission unit according to claim 3,
wherein the monitoring cylinder is configured as a peripheral cylindrical body integrally formed on an outer periphery of the housing.

9. The dental instrument with the built-in camera according to claim 7, wherein the arrangement of mounting of the wireless transmission unit to the housing of the dental instrument with the built-in camera is such that when an assumed reference line extending in a projecting direction from the housing of the tool is assumed, the wireless transmission unit is arranged to be inclined by a predetermined angle to the right in the case of right-handed specifications and to the left in the case of left-handed specifications with respect to the assumed reference line as viewed from the distal end side of the housing.

10. The dental instrument with the built-in camera according to claim 7, wherein each of the destination apparatuses is selected from a dental care unit, cart type image display, a personal computer or a head mounted display which respectively have a receiving transmitter.

11. The dental instrument with the built-in camera according to claim 7, which is selected from an air turbine hand piece, a micromotor hand piece, a syringe or a scaler which respectively have incorporated a camera therein.

12. The wireless transmission unit for use in the dental instrument with the built-in camera according to claim 2, wherein the arrangement of mounting of the wireless transmission unit to the housing of the dental instrument with the built-in camera is such that when an assumed reference line extending in a projecting direction of the tool from the housing is assumed, the wireless transmission unit is arranged to be inclined by a predetermined angle to the right in the case of right-handed specifications and to the left in the case of left-handed specifications with respect to the assumed reference line as viewed from the distal end side of the housing.

13. The wireless transmission unit for use in the dental instrument with the built-in camera according to claim 3, wherein the arrangement of mounting of the wireless transmission unit to the housing of the dental instrument with the built-in camera is such that when an assumed reference line extending in a projecting direction of the tool from the housing is assumed, the wireless transmission unit is arranged to be inclined by a predetermined angle to the right in the case of right-handed specifications and to the left in the case of left-handed specifications with respect to the assumed reference line as viewed from the distal end side of the housing.

14. The wireless transmission unit for use in the dental instrument with the built-in camera according to claim 2, wherein each of the destination apparatuses is selected from a dental care unit, cart type image display, a personal computer or a head mounted display which respectively have a receiving transmitter.

15. The wireless transmission unit for use in the dental instrument with the built-in camera according to claim 3, wherein each of the destination apparatuses is selected from a dental care unit, cart type image display, a personal computer or a head mounted display which respectively have a receiving transmitter.

16. The wireless transmission unit for use in the dental instrument with the built-in camera according to claim 2, wherein the dental instrument with the built-in camera is selected from an air turbine hand piece, a micromotor hand piece, a syringe or a scaler which respectively have incorporated a camera therein.

17. The wireless transmission unit for use in the dental instrument with the built-in camera according to claim 3, wherein the dental instrument with the built-in camera is selected from an air turbine hand piece, a micromotor hand piece, a syringe or a scaler which respectively have incorporated a camera therein.

18. The dental instrument with the built-in camera according to claim 8, wherein the arrangement of mounting of the wireless transmission unit to the housing of the dental instrument with the built-in camera is such that when an assumed reference line extending in a projecting direction from the housing of the tool is assumed, the wireless transmission unit is arranged to be inclined by a predetermined angle to the right in the case of right-handed specifications and to the left in the case of left-handed specifications with respect to the assumed reference line as viewed from the distal end side of the housing.

19. The dental instrument with the built-in camera according to claim 8, wherein each of the destination apparatuses is selected from a dental care unit, cart type image display a personal computer or a head mounted display which respectively have a receiving transmitter.

20. The dental instrument with the built-in camera according to claim 8, which is selected from an air turbine hand piece, a micromotor hand piece, a syringe or a scaler which respectively have incorporated a camera therein.
